(19) United States
(12) Patent Application Publication
(10) Pub. No.: US 2026/0124443 A1
(43) Pub. Date: May 7, 2026

(54) TEMPORARY PACING LEAD MANAGEMENT SYSTEM WITH EXPANDABLE MEMBER

(71) Applicant: Medtronic, Inc., Minneapolis, MN (US)

(72) Inventors: Leah M. Severseike, Phoenix, AZ (US); Kevin R. Seifert, Forest Lake, MN (US); Jonathan A. Hughes, Blaine, MN (US); Mikayle A. Holm, Saint Anthony, MN (US); Teresa A. Whitman, Dayton, MN (US); Thomas A. Wong, Little Canada, MN (US)

(21) Appl. No.: 19/114,736

(22) PCT Filed: Sep. 7, 2023
(86) PCT No.: PCT/IB2023/058876

§ 371 Date: Mar. 24, 2025

Related U.S. Application Data
(60) Provisional application No. 63/377,151, filed on Sep. 26, 2022.

(51) Int. Cl. A61N 1/05

(52) U.S. Cl. CPC A61N 1/057; A61N 1/056

(57) ABSTRACT

This disclosure describes a system comprising an electrical lead, an implantable medical device, wherein the electrical lead is configured to be electrically connected to the implantable medical device, and wherein the implantable medical device is configured to deliver an electrical therapy to tissue of a patient via the electrical lead, and an expandable member configured to be disposed over the implantable medical device and an excess portion of the electrical lead. The expandable member comprises a first portion defining an inner volume configured to retain the implantable medical device and the excess portion of the electrical lead, and a second portion connected to a proximal end of the first portion, the second portion configured to be disposed over at least a part of the first portion of the expandable member, wherein the expandable member is configured to control a length of the electrical lead within vasculature of the patient.

This application claims the benefit of U.S. Provisional Patent Application Ser. No. 63/377,151, filed 26 Sep. 2022, the entire content of which is incorporated herein by reference.

### TECHNICAL FIELD

This disclosure relates to medical device systems and, more particularly, to medical device systems for delivery of electrical stimulation therapy.

### BACKGROUND

In some situations, medical professionals may insert an implantable medical lead into the body of the patient to deliver various therapies. In some examples, the implantable medical leads may be temporary and may be removed from the patient after a period of time. In some examples, with respect to cardiac-related issues, a medical professional may insert a type of implantable medical lead also referred to as a temporary pacing lead into a heart of the patient and deliver temporary cardiac pacing to the heart.

### SUMMARY

The devices, systems, and techniques of this disclosure generally relate to managing movement of an implantable medical lead within a patient. In some examples, the implantable medical lead may have an excess portion that may be placed near an implantable medical device connected to the implantable medical lead. A clinician may need to control movement of the implantable medical lead within the patient. In some examples, the clinician may need to remove the implantable medical device and the implantable medical lead from the patient. This disclosure describes devices, systems, and techniques for managing movement of the implantable medical lead within the patient and devices, systems, and techniques for facilitating removal of the implantable medical lead from the patient.

In an example, the disclosure describes a system comprising: an electrical lead; an implantable medical device, wherein the electrical lead is configured to be electrically connected to the implantable medical device, and wherein the implantable medical device is configured to deliver an electrical therapy to tissue of a patient via the electrical lead; and an expandable member configured to be disposed over the implantable medical device and an excess portion of the electrical lead, the expandable member comprising: a first portion defining an inner volume configured to retain the implantable medical device and the excess portion of the electrical lead; and a second portion connected to a proximal end of the first portion, the second portion configured to be disposed over at least a part of the first portion of the expandable member, wherein the expandable member is configured to control a length of the electrical lead within vasculature of the patient.

In another example, the disclosure describes a method comprising: inserting a distal portion of an electrical lead of a medical device system into vasculature of a patient, wherein the medical device system comprises: an implantable medical device electrically connected to the electrical lead; an expandable member comprising: a first portion defining an inner volume; and a second portion connected to a proximal end of the first portion; navigating the distal portion of the electrical lead to a target location within the vasculature of the patient; implanting the distal portion of the electrical lead in tissue at the target location; disposing the implantable medical device and an excess portion of the electrical lead within the inner volume of the expandable member; disposing the second portion of the expandable member over at least a part of the first portion of the expandable member; and delivering electrical therapy to the tissue at the target location through the electrical lead.

In another example, the disclosure describes a lead retention device comprising: an expandable member comprising: a first portion defining an inner volume, wherein the inner volume is configured to retain an implantable medical device and an excess portion of an electrical lead; and a second portion connected to a proximal end of the first portion wherein the second portion is configured to be disposed over at least a part of the first portion of the expandable member, wherein the lead retention device is configured to control a length of the electrical lead within vasculature of a patient.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a conceptual diagram illustrating an implantable medical device system including a lead management system.

FIG. 2 is a block diagram illustrating an example configuration of an implantable medical device of the system of FIG. 1.

FIG. 3A is a conceptual diagram illustrating an example expandable member of a lead management system in a deployed configuration.

FIG. 3B is a conceptual diagram illustrating the example expandable member of FIG. 3A in a wrapped configuration.

FIGS. 4A-D are conceptual diagrams illustrating an example process of deploying the example expandable member of FIG. 3A.

FIG. 5 is a flowchart illustrating an example process of implanting an expandable member and an implantable medical device in a patient.

### DETAILED DESCRIPTION

Medical devices, systems, and techniques of this disclosure relates to the managing movement of an implantable medical lead within a patient and facilitating removal of the implantable medical lead from the patient. A medical professional may insert an implantable medical lead into the patient (e.g., through vasculature of the patient) to deliver electrical signals (e.g., for purposes of medical therapy, stimulation, or the like) to tissue of the patient. The implantable medical lead (also referred to herein as “electrical lead”) may be connected to an implantable medical device. The implantable medical device may generate the electrical signals and transmit the electrical signals to the tissue of the patient through one or more implantable medical leads.

In some examples, based on the anatomy of the patient and/or the target location, the length of the implantable medical lead may be longer than necessary to reach the target site from the implantable medical device and the clinician may store the excess lead length in subcutaneous tissue of the patient (e.g., adjacent to the implantable medical device within the subcutaneous tissue). Storage of the excess lead length in the tissue of the patient may lead to twisting, crimping, and/or pinching of the implantable medical lead which may negatively affect performance of the lead. In some examples, tissue may grow around the implantable medical device and/or the excess lead lengths within the subcutaneous tissue and renders removal of the implantable medical device and/or the implantable medical lead difficult and time consuming.

This disclosure provides improvements over other implantable medical lead systems and insertion processes by allowing a clinician to manage the length of an implantable medical lead within the vasculature of the patient and/or to manage movement of the implantable medical lead within a subcutaneous pocket in the tissue of the patient and/or within the vasculature of the patient. The medical devices, systems, and techniques of this disclosure also isolates the excess portions of the implantable medical lead stored in the subcutaneous pocket from tissue of the patient and facilitate removal of the implantable medical device and the implantable medical lead during a later operation. The medical devices, systems, and techniques of this disclosure may also enable identification of an orientation of the distal end of the implantable medical lead in addition to its position.

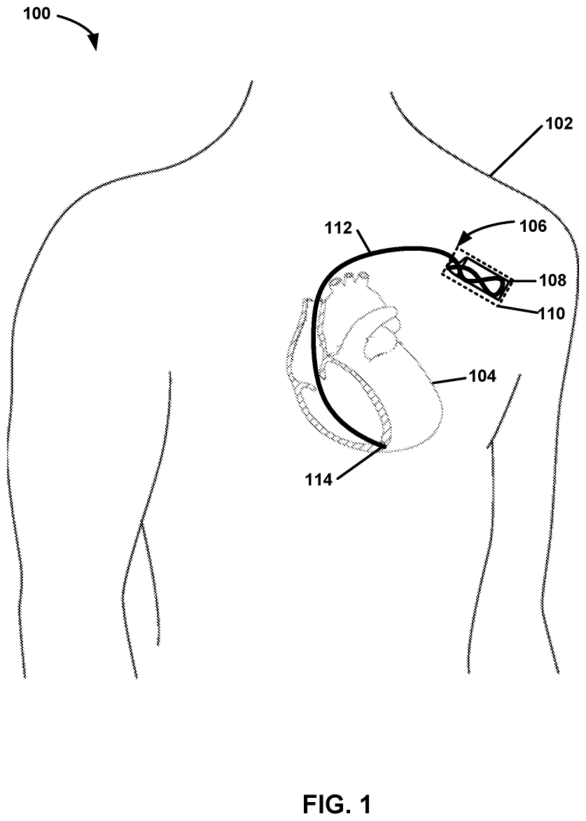

FIG. 1 is a conceptual diagram illustrating an implantable medical device system 100 including a lead management system 106. Medical device system 100 may include implantable medical device (IMD) 108, lead management system 106 including lead management device 110, and implantable medical lead 112 (also referred to herein as “electrical lead 112”). In some examples, as illustrated in FIG. 1, at least parts of medical device system 100 (e.g., IMD 108, lead management system 106, and electrical lead 112.) may be implanted within patient 102 (e.g., within a subcutaneous pocket in patient 102). A distal end 114 of lead 112 may be implanted at a target location (e.g., within heart 104 as illustrated in FIG. 1) within patient 102.

IMD 108 may include one or more devices configured to generate and deliver electrical energy (e.g., electrical stimulation, electrical therapy, or the like) to tissue of patient 102. The IMD 108 may be implanted within a subcutaneous pocket in patient 102. The subcutaneous pocket may be outside vasculature of patient 102. IMD 108 may deliver electrical energy to one or more target locations within patient 102 via one or more electrical leads 112. IMD 108 may be an implantable pulse generator (IPG), an implantable cardioverter defibrillator (ICD), a cardiac resynchronization therapy (CRT) device, or the like.

Electrical lead 112 may be connected to IMD 108 (e.g., to a distal end of IMD 108). Electrical lead 112 may transmit electrical signals between tissue of patient 102 and IMD 108. In some examples, as described in greater detail below, medical device system 100 may include a single electrical lead 112. In other examples, medical device system 100 may have two or more electrical leads 112. A clinician may insert electrical lead 112 into patient 102 at an insertion site (not pictured). The insertion site may be in the upper thorax or clavicular area of patient 102. In other examples, the insertion site may be at other locations on patient 102 (e.g., the groin, the neck, the abdomen, the arm, and the like). The medical professional may insert distal portion 114 of electrical lead 112 into vasculature of patient 102 at the insertion site and navigate electrical lead 112 to a target location (also referred to herein as “target treatment site”) within patient 102 through vasculature of patient 102 (e.g., within heart 114).

The clinician may navigate distal portion 114 of electrical lead 112 through the vasculature of patient 102 using one or more imaging techniques (e.g., X-ray imaging, fluoroscopy, ultrasound, or the like). Once the clinician determines that distal portion 114 of electrical lead 112 is at the target location, the clinician may implant the distal portion 114 at the target location via one or more fixation mechanisms attached to distal portion 114 of electrical lead 112. Depending on the anatomy of patient 102 and/or distance within the vasculature between the target location and the insertion site, a proximal portion of electrical lead 112 (also referred to as “excess portion”) may be outside of the vasculature of patient 102.

Lead management system 106 includes lead management device 110 which may be disposed over IMD 108 and electrical lead 112 (e.g., the excess portion of electrical lead 112). Lead management device 110 (also referred to as “expandable member 110”) may be an expandable material and may control (e.g., inhibit or prevent) movement of electrical lead 112 within the vasculature of patient 102 by retention of the excess portion of electrical lead 112. Lead management device 110 containing IMD 108 and electrical lead 112 may be implanted within a subcutaneous pocket at the insertion site. While implanted, lead management device 110 may isolate IMD 108 and/or the excess portions of electrical lead 112 from tissue of patient 102 around the subcutaneous pocket and prevent tissue ingrowth around IMD 108 and/or electrical lead 112.

Expandable member 110 may include a first portion and a second portion connected to a proximal end of the first portion. The first portion and the second portion of expandable member 110 may define an inner volume. IMD 108 and the excess portion of electrical lead 112 may be disposed within the inner volume of expandable member 110. In some examples, the clinician may dispose IMD 108 and the excess portion of electrical lead 112 within the first portion of expandable member 110. The clinician may then dispose the second portion of expandable member 110 over at least a part of the first portion of expandable member 110, e.g., to isolate IMD 108 and the excess portion of electrical lead 112 from tissue of patient 102. In some examples, expandable member 110 may retain the excess portion of electrical lead 112 in a storage configuration.

In some examples, lead management system 106 may include an attachment member (e.g., an attachment sheath) connected to a distal end of expandable member 110. The attachment member may be at last partially disposed within the vasculature of patient 102. The attachment member may anchor electrical lead 112 within the vasculature of patient 102.

Examples of the sheath are described in the U.S. Provisional Patent Application entitled “Temporary Pacing Lead Management System”, having Attorney Docket Number A0007885US01/2222-205USP1 and filed on the same day as this disclosure, the entirety of which is incorporated by reference herein.

When the clinician decides to remove IMD 108 and/or electrical lead 112 from patient 102, the clinician may re-open the subcutaneous pocket and remove lead management device 110 containing IMD 108 and electrical lead 112 without necessitating removal of ingrown tissue around IMD 108 and/or electrical lead 112. In some examples, electrical lead 112 may remain implanted within patient 102 and the clinician may replace IMD 108 of medical device system 100 with another implantable medical device. In some examples, expandable member 110 may remain implanted within tissue of patient 102 and the clinician may remove IMD 108 and/or electrical lead 112 from within patient 102, e.g., by removing the second portion of expandable member 110 from over at least a part of the first portion of expandable member 110 to allow access to the inner volume of expandable member 110.

Medical system 100 may also include one or more computing devices and/or systems (not pictured) in communications with IMD 108. The one or more computing devices and/or systems may be operated by a user (e.g., by patient 102 and/or the clinician) and may transmit instructions to IMD 108. In some examples, the one or more computing devices and/or systems may receive patient information detected by IMD 108 (e.g., via electrical lead 112).

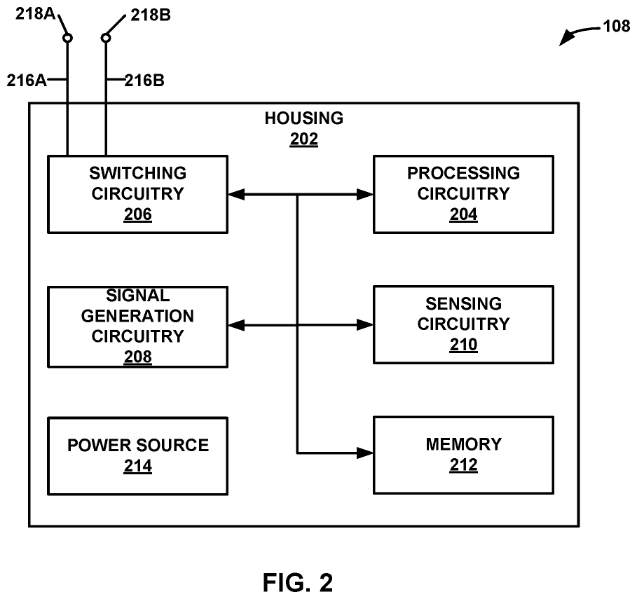

FIG. 2 is a block diagram illustrating an example configuration of an IMD 108 of the medical device system 100 of FIG. 1. IMD 108 may include processing circuitry 204, switching circuitry 206, signal generation circuitry 208, sensing circuitry 210, memory 212, power source 214, and electrodes 218A-B (also referred to as “electrodes 218”) connected to conductors 216 A-B (also referred to as “conductors 216”). Conductors 216 and electrodes 218 may be disposed within electrical lead 112 (not pictured). While FIG. 2 illustrates IMD 108 having two electrodes 218, other example IMDs may include three or more electrodes, each electrode connected to a corresponding conductor. In some examples, IMD 108 may have additional components (e.g., communications circuitry or the like). In some examples, as illustrated in FIG. 2, the components of IMD 108 may be contained within housing 202. In some examples electrodes 218 may be disposed in distal end 114 of electrical lead 112.

The various circuitry may be, or include, programmable or fixed function circuitry configured to perform the functions attributed to respective circuitry. Memory 212 may store computer-readable instructions that, when executed by processing circuitry 204, cause computing system 112 and/or implantable medical lead 104 to perform various functions. Memory 212 may be a storage device or other non-transitory medium.

Signal generation circuitry 208 is configured to generate electrical signals (e.g., stimulation signals such as cardiac pacing pulses). Signal generation circuitry 208 may include, as examples, current or voltage sources, capacitors, charge pumps, or other signal generation circuitry. Switching circuitry 206 is coupled to electrodes 218 and may include one or more switch arrays, one or more multiplexers, one or more switches (e.g., a switch matrix or other collection of switches), one or more transistors, or other electrical circuitry. Switching circuitry 206 is configured to direct electrical signals from signal generation circuitry 208 to a selected combination of electrodes 218, having selected polarities, e.g., to selectively deliver electrical signals to one or more target locations within patient 102. In some examples, signal generation circuitry 208 and switching circuitry 206 may be configured to selectively deliver electrical signals to one or more chambers of heart 104 of patient 102 to deliver cardiac pacing pulses to heart 104.

Switching circuitry 206 may also selectively couple sensing circuitry 210 to selected combinations of electrodes 218 to selectively sense the electrical activity in one or more target locations within patient 102. Sensing circuitry 210 may include filters, amplifiers, analog-to-digital converts, or other circuitry configured to sense electrical signals (e.g., cardiac electrical signals) via electrodes 218. For example, switching circuitry 206 may couple each of electrodes 218 to respective sensing channels provided by sensing circuitry 210 to sense the electrical signals in the corresponding target locations. Sensing circuitry 210 may sense electrical signals via electrodes 218 and store the sensed electrical signals within memory 212.

Processing circuitry 204 may include any one or more of a microprocessor, a controller, a digital signal processor (DSP), an application specific integrated circuitry (ASIC), a field-programmable gate array (FPGA), discrete logic circuitry, or any other processing circuitry configured to provide the functions attribute to processing circuitry 204 herein may be embodied as firmware, hardware, software, or any combination thereof.

Processing circuitry 204 may retrieve and execute instructions from memory 212 and transmit instructions to switching circuitry 206, signal generation circuitry, and sensing circuitry 210 to perform the functions described above.

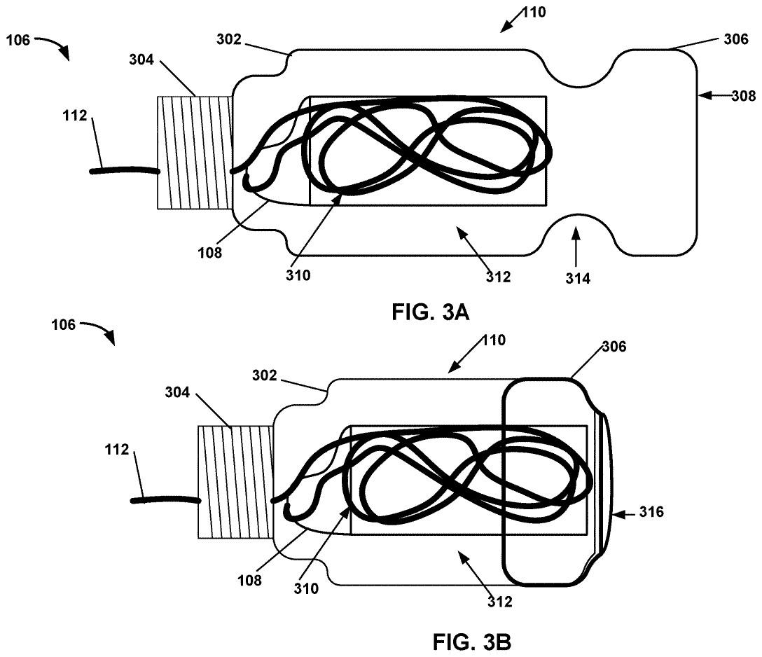

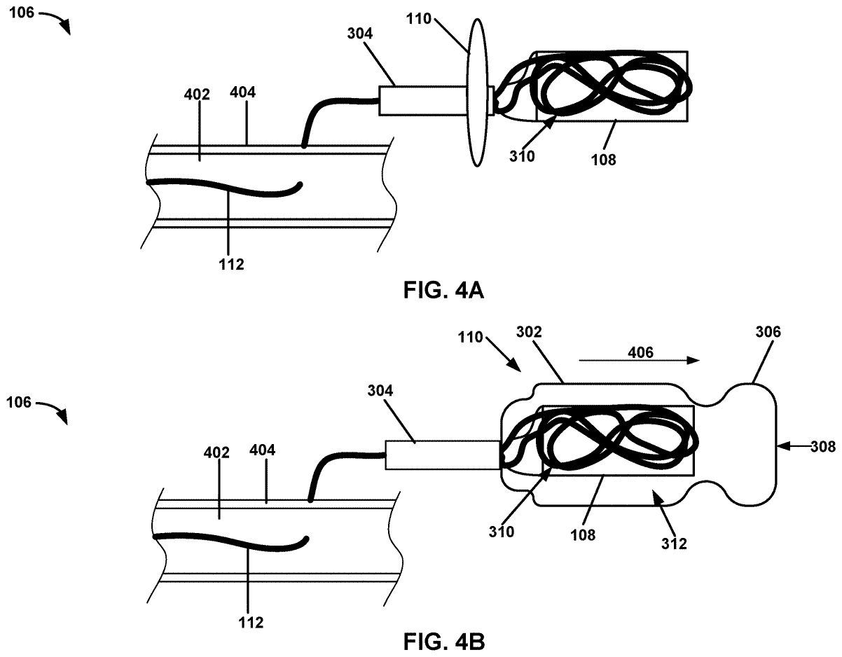

FIGS. 3A and 3B illustrate an example expandable member 110. FIG. 3A is a conceptual diagram illustrating expandable member 110 of lead management system 106 in a deployed configuration. Expandable member 110 may include first portion 302 and second portion 306. A distal end of first portion 302 may be connected to attachment member 304. IMD 108 and excess portion 310 of electrical lead 112 may be disposed within expandable member 110. FIG. 3B is a conceptual diagram illustrating expandable member 110 in a wrapped configuration.

As illustrated in FIG. 3A, second portion 306 may be connected to a proximal end of first portion 302. Expandable member 110 may define inner volume 312 which may be accessed by opening 308 defined by second portion 306. Inner volume 312 may retain IMD 108 and/or electrical lead 112 of varying sizes. In some examples, expandable member 110 may isolate IMD 108 and/or electrical lead 112 from tissue of patient 102.

In some examples, the clinician may dispose IMD 108 and excess portion 310 into inner volume 312 while expandable member 110 is in the deployed configuration as shown in FIG. 3A. In other examples, the clinician may roll expandable member 110 from a pre-deployed configuration into the deployed configuration and over IMD 108 and excess portion 310 of electrical lead 112.

Lead management system 106 may include attachment member 304 connected to expandable member 110 (e.g., to distal end of first portion 302). In some examples, a distal end of attachment member 304 may be connected to IMD 108, electrical lead 112, and/or one or more other devices of medical device system 100 (e.g., a guide sheath disposed within the vasculature of patient 102). In some examples, attachment member 304 may be at least partially disposed within the vasculature. While disposed within the vasculature, attachment member 304 may be configured to anchor expandable member 110 and/or electrical lead 112 within the vasculature.

In some examples, the clinician may first arrange excess portion 310 into a storage configuration (e.g., as illustrated in FIG. 3A) before inserting electrical lead 112 into and through expandable member 110. In other examples, the clinician may insert electrical lead 112 into and through expandable member 110 before arranging excess portion 310 of electrical lead 112 into the storage configuration. While in the storage configuration, one or more coils of electrical lead 112 may not be pinched, twisted, crimped, or the like.

Expandable member 110 includes an expandable material configured to expand around and IMD 108 and excess portion 306. In some examples, the expandable material may include a biocompatible polymer including silicone, latex, polyurethane, or the like. Expandable member 110 may be transparent or translucent. The transparency of the expandable member 110 may allow the clinician to determine the positioning of IMD 108 and excess portion 306 within expandable member 110 and the presence of any twisting and/or crimping of any portion of electrical lead 112.

In some examples, expandable member 110 may include a relatively lubricious material disposed around an outer surface of expandable member 110. The lubricious material may increase the ease of insertion and/or extraction of expandable member 110 from the subcutaneous pocket by the clinician during medical device system 100 implantation and/or removal procedures. The lubricious material may include, but is not limited to, silicone oil, Polytetrafluoroethylene (PTFE), a calcium carbonate coating, or Ethylene tetrafluoroethylene (ETFE). Expandable member 110 may also include an anti-infection material disposed on an inner surface of expandable member 110. The anti-infection material may reduce adverse reactions by patient 102 to implantation of IMD 108, expandable member 110, and electrical lead 112 within tissue of patient 102. In some examples, the anti-infection material includes, but is not limited to, antimicrobial agents such as minocycline and/or rifampin. Expandable member 110 may include an anti-bleeding and anti-coagulation material. In some examples, the anti-bleeding and anti-coagulation material may be disposed over an outer surface of expandable member 110. The anti-bleeding and anti-coagulation material may include but are not limited to, Alginate, or Tranexamic Acid.

As illustrated in FIG. 3B, second portion 306 may twist about a longitudinal axis of expandable member 110 and may be disposed over at least a part of first portion 302. When second portion 306 is disposed over first portion 302, second portion 306 may wrap around a proximal portion (e.g., a proximal end) of first portion 302 and may seal inner volume 312 from tissue of patient 102. Second portion 306 may form seal 316 around proximal end of first portion 302.

In some examples, the clinician may use one or more fixation devices (e.g., a suture or like) to seal second portion 306 around first portion 302. In other examples, as illustrated in FIG. 3B, second portion 306 may seal around first portion 302 without requiring the use of other fixation devices. In some examples, the outer surface of first portion 302 may include one or more features (e.g., indentations or the like) that may affix to second portion 306 as second portion 306 wraps around first portion 302 to seal second portion 306 around first portion 302.

First portion 302 and/or second portion 306 may define indentations 314. Indentations 314 may allow for twisting of second portion 306 about a longitudinal axis of expandable member 110 and for to wrap second portion 306 around at least a part of first portion 302.

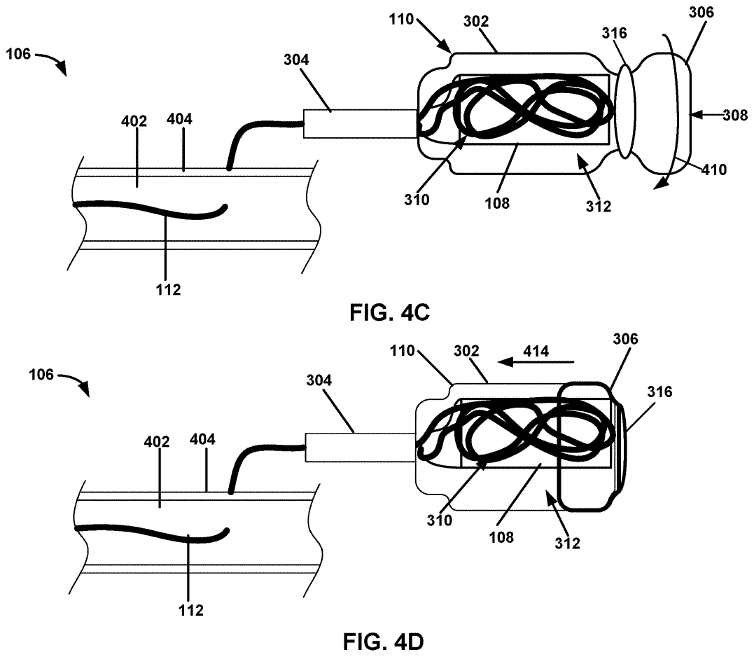

FIGS. 4A-D is a conceptual diagram illustrating an example process of deploying expandable member 110 of FIG. 3A. FIG. 4A illustrates lead management system 106 with expandable member 110 in a pre-deployed configuration. FIG. 4B illustrates lead management system 106 with expandable member 110 in a deployed configuration. FIG. 4C illustrates second portion 306 of expandable member 110 in a twisted configuration. FIG. 4D illustrates second portion 306 disposed over at least part of first portion 302.

In some examples, as illustrated in FIG. 4A, attachment member 304 may be connected to IMD 108. Electrical lead 112 may be disposed within an inner lumen defined by attachment member 304 and into blood vessel 402. In some examples, attachment member 304 may penetrate vessel wall 404 and dispose electrical lead 112 within blood vessel 402. In other examples, as illustrated in FIG. 4A, attachment member 304 may be wholly outside blood vessel 402 and the clinician may insert electrical lead 112 into blood vessel 402 by making an incision in vessel wall 404.

Expandable member 110 may be disposed on attachment member 304. Expandable member 110 may be permanently and/or removably connected to attachment member 304 and/or IMD 108. In some examples, as illustrated in FIG. 4A, expandable member 110 may be disposed in a pre-deployed configuration. Expandable member 110 may be positioned distal to a distal end of IMD 108 and excess portion 310 of electrical lead 112. Prior to deployment of expandable member 110, the clinician may position excess portion 310 of electrical lead 112 (e.g., in a storage configuration) next to IMD 108. In some examples, IMD 108 may include one or more attachment members disposed on outer housing 202 and configured to secure excess portion 310 to IMD 108.

As illustrated in FIG. 4B, the clinician may apply force 406 to deploy expandable member 110 to the deployed configuration. Expandable member 110 may expand along a longitudinal axis of IMD 108 and/or radially outwards of IMD 108 as the clinician deploys expandable member 110. When expandable member 110 is fully deployed, IMD 108 and excess portion 306 may be fully disposed within inner volume 312 of expandable member 110 (e.g., entirely within first portion 302 of expandable member 110). As the clinician deploys expandable member 110, the clinician may dispose IMD 108 and excess portion 310 within inner volume 312 via opening 308.

As illustrated in FIG. 4C, once IMD 108 and excess portion 310 are fully disposed within inner volume 312, the clinician may apply force 308 to twist second portion 306 around a longitudinal axis of expandable member 110. By twisting second portion 306, the clinician may form seal 316 between first portion 302 and second portion 306. Seal 316 may seal off inner volume 312 between first portion 302 and second portion 306 and isolate first portion 302 from tissue of patient 102. Force 410 may be in the clockwise direction, in the counter-clockwise direction, or in either direction. In some examples, second portion 306 may be disposed over first portion 302 and form seal 316 without requiring the clinician to twist second portion 306.

As illustrated in FIG. 3D, the clinician may dispose second portion 306 over at least a part of first portion 302, e.g., to wrap around the part of first portion 302. When disposed over the part of first portion 302, second portion 306 and/or seal 316 may isolate IMD 108 and excess portion 310 from tissue of patient 102. The clinician may apply force 414 on second portion 306 to dispose second portion 306 over the part of first portion 302. Force 414 may be in a direction towards distal end of expandable member 110 and/or towards attachment member 304. In some examples, after disposing second portion 306 over first portion 302, the clinician may secure second portion 306 to first portion 302 with one or more fixation devices. In other examples, as illustrated in FIG. 4D, second portion 306 may be automatically secured to first portion 302.

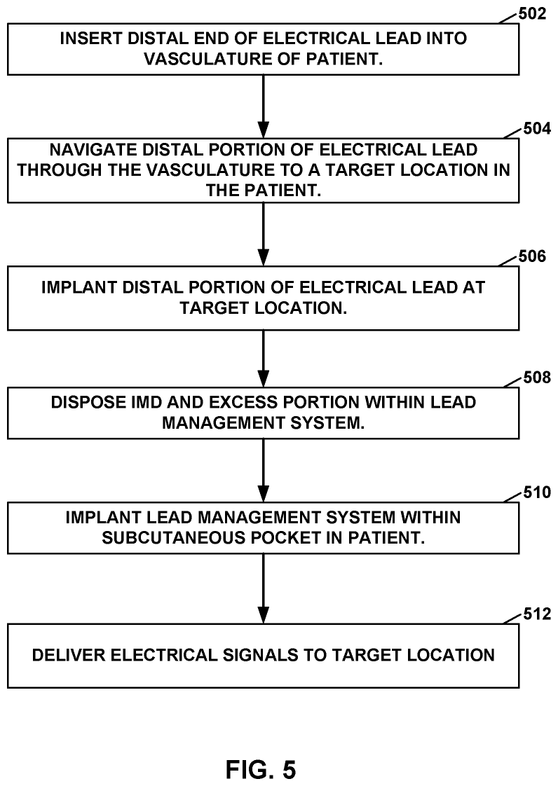

FIG. 5 is a flowchart illustrating an example process of implanting an expandable member and an implantable medical device in a patient. A clinician may insert distal end 114 of an electrical lead 112 into vasculature of patient 102 (502). The clinician may make an insertion at an insertion site on patient 102. The insertion may extend from the skin of patient 102 into vasculature of patient 102, e.g., via a blood vessel of patient 102. Once the clinician makes the incision, the clinician may insert distal end 114 of electrical lead 112 through the incision and into the vasculature.

The clinician may navigate electrical lead 112 through the vasculature to a target location (also referred to as “target treatment site”) within patient 102 (504). The target location may be any location within vasculature of patient 102 that the clinician wants to deliver electrical energy to. In some examples, the target location may include wall tissue (e.g., at the Triangle of Koch) of one or more chambers of heart 104 of patient 102. The clinician may navigate electrical lead 112 to the target location via one or more imaging techniques (e.g., X-ray imaging, fluoroscopy, ultrasound, or the like). In some examples, the clinician may navigate electrical lead 112 via one or more radiopaque markers disposed on electrical lead 112.

The clinician may implant distal end 114 of electrical lead 114 at the target location (506). The clinician may implant distal end 114 of electrical lead 112 via one or more fixation features disposed on distal end 114. Fixation features may include, but are not limited, penetrator(s) (e.g., a helical penetrator), tines, adhesives, or the like.

The clinician may dispose IMD 108 and excess portion 310 of electrical lead 112 within lead management system 106 (508). Lead management system 106 includes lead management device 110 such as expandable member 110. The clinician may dispose IMD 108 and excess portion 310 within an inner volume 312 of expandable member 110. The clinician may arrange excess portion 310 into a storage configuration before placing excess portion 310 into inner volume 312. The clinician may inspect expandable member 110 to determine if excess portion 310 is disposed within inner volume 312 such that a portion of excess portion 306 is twisted, crimped, pinched, or the like. Based on a determination that excess portion 306 is twisted, crimped, pinched, or the like, the clinician may reposition excess portion 306 within inner volume 312 and/or reinsert IMD 108 and/or excess portion 310 into expandable member 110.

The clinician may implant lead management system 106 within a subcutaneous pocket in patient 102 (510). In some examples, the clinician may implant expandable member 110 within the subcutaneous pocket after disposing IMD 108 and excess portion 310 within inner volume 312 of expandable member 110. In other examples, the clinician may implant expandable member 110 within the subcutaneous pocket as a part of disposing IMD 108 and excess portion 310 within the subcutaneous pocket. In some examples, the clinician may create the subcutaneous pocket and then dispose expandable member 110 within the subcutaneous pocket. In other examples, the clinician may create the subcutaneous pocket as a part of disposing expandable member 110 within tissue of patient 102.

The clinician may deliver electrical signals to target location (512). In some examples, the clinician may instruct IMD 108 to deliver electrical signals to target location via electrical lead 112 after implantation of IM 108 and electrical lead 112 within tissue of patient 102. In some examples, after implantation of IMD 108, IMD 108 may automatically transmit electrical signals to the target location.

The techniques of this disclosure may be implemented in a wide variety of computing devices, medical devices, or any combination thereof. Any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis.

The techniques described in this disclosure, including those attributed to various modules and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated, discrete logic circuitry, or other processing circuitry, as well as any combinations of such components, remote servers, remote client devices, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. For example, any module described herein may include electrical circuitry configured to perform the features attributed to that particular module, such as fixed function processing circuitry, programmable processing circuitry, or combinations thereof.

The techniques described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media. The computer-readable storage medium may also be referred to as storage devices.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

It should be noted that medical device system 100, and the techniques described herein, may not be limited to use in a human patient. In alternative examples, medical device system 100 may be implemented in non-human patients, e.g., primates, canines, equines, pigs, and felines. These other animals may undergo clinical or research therapies that my benefit from the subject matter of this disclosure. Various examples are described herein, such as the following examples.

Example 1: a system comprising: an electrical lead; an implantable medical device, wherein the electrical lead is configured to be electrically connected to the implantable medical device, and wherein the implantable medical device is configured to deliver an electrical therapy to tissue of a patient via the electrical lead; and an expandable member configured to be disposed over the implantable medical device and an excess portion of the electrical lead, the expandable member comprising: a first portion defining an inner volume configured to retain the implantable medical device and the excess portion of the electrical lead; and a second portion connected to a proximal end of the first portion, the second portion configured to be disposed over at least a part of the first portion of the expandable member, wherein the expandable member is configured to control a length of the electrical lead within vasculature of the patient.

Example 2: the system of example 1, wherein the expandable member is configured to control the length of the electrical lead by securing the excess portion of the electrical lead within the inner volume in a storage configuration.

Example 3: the system of any of examples 1 and 2, wherein the expandable member is configured to isolate the implantable medical device and the excess portion of the electrical lead from the tissue of the patient.

Example 4: the system of example 3, wherein the second portion of the expandable member is configured to isolate the inner volume of the expandable member from the tissue of the patient when disposed over the first portion.

Example 5: the system of any of examples 1-4, wherein the expandable member is configured to roll over the implantable medical device from a distal end of the implantable medical device to a proximal end of the implantable medical device.

Example 6: the system of any of examples 1-5, wherein the second portion of the expandable member is configured to be disposed over the first portion through twisting of the second portion relative to the first portion and rolling of the second portion over the first portion.

Example 7: the system of any of examples 1-6, wherein the expandable member comprises silicone.

Example 8: the system of any of examples 1-6, wherein the expandable member comprises a biocompatible polymer.

Example 9: the system of any of examples 1-8, wherein the expandable member further comprises an attachment member connected to a distal end of the first portion, wherein the attachment member is configured to secure the expandable member to at least one of the implantable medical device or the electrical lead.

Example 10: the system of example 9, wherein the attachment member is further configured to anchor the electrical lead within the vasculature of the patient.

Example 11: the system of any of examples 1-10, wherein the expandable member further comprises an anti-infection material.

Example 12: the system of any of examples 1-11, wherein the expandable member is configured to be implanted in a subcutaneous pocket outside of the vasculature of the patient.

Example 13: the system of any of examples 1-12, wherein the expandable member is self-sealing.

Example 14: the system of any of examples 1-13, wherein the expandable member comprising an anti-bleeding and anti-coagulation material.

Example 15: a method comprising: inserting a distal portion of an electrical lead of a medical device system into vasculature of a patient, wherein the medical device system comprises: an implantable medical device electrically connected to the electrical lead; an expandable member comprising: a first portion defining an inner volume; and a second portion connected to a proximal end of the first portion; navigating the distal portion of the electrical lead to a target location within the vasculature of the patient; implanting the distal portion of the electrical lead in tissue at the target location; disposing the implantable medical device and an excess portion of the electrical lead within the inner volume of the expandable member; disposing the second portion of the expandable member over at least a part of the first portion of the expandable member; and delivering electrical therapy to the tissue at the target location through the electrical lead.

Example 16: the method of example 15, wherein reducing the length of the electrical lead to the desired length comprises: determining the desired length and the excess portion of the electrical lead; securing the excess portion of the electrical lead into a storage configuration; and storing the excess portion of the electrical lead in the inner volume of the expandable member in the storage configuration.

Example 17: the method of any of examples 15 and 16, wherein the expandable member isolates the implantable medical device and the excess portion of the electrical lead from the tissue of the patient.

Example 18: the method of example 17, wherein the second portion of the expandable member, when disposed over the first portion, isolates the inner volume from the tissue.

Example 19: the method of any of examples 15-18, wherein disposing the implantable medical device and the excess portion of the electrical lead within the inner volume of the expandable member comprises rolling the expandable member over the implantable medical device from a distal end of the implantable medical device to a proximal end of the implantable medical device.

Example 20: the method of any of examples 15-19, wherein disposing the second portion of the expandable member over the first portion of the expandable member comprises: twisting the second portion relative to the first portion; and roll the second portion over the first portion from the proximal end of the first portion towards a distal end of the first portion.

Example 21: the method of any of examples 15-20, wherein the expandable member comprises silicone.

Example 22: the method of any of examples 15-21, wherein the expandable member comprises a biocompatible polymer.

Example 23: the method of any of examples 15-22, wherein the expandable member further comprises an attachment member connected to a distal end of the first portion, the attachment member securing the expandable member to at least one of the implantable medical device or the electrical lead.

Example 24: the method of example 23, further comprising, securing the electrical lead to the vasculature of the patient via the attachment member of the expandable member.

Example 25: the method of any of examples 15-24, wherein the expandable member further comprises an anti-infection material.

Example 26: the method of any of examples 15-25, wherein the expandable member is self-sealing.

Example 27: the method of any of examples 15-26, wherein the expandable member comprises an anti-bleeding and anti-coagulation material.

Example 28: the method of any of examples 15-27, wherein disposing the implantable medical device and the excess portion of the electrical lead within the inner volume of the expandable member comprises: disposing the excess portion of the electrical lead within the inner volume without twisting the electrical lead.

Example 29: a lead retention device comprising: an expandable member comprising: a first portion defining an inner volume, wherein the inner volume is configured to retain an implantable medical device and an excess portion of an electrical lead; and a second portion connected to a proximal end of the first portion, wherein the second portion is configured to be disposed over at least a part of the first portion of the expandable member, wherein the lead retention device is configured to control a length of the electrical lead within vasculature of a patient.

Example 30: the device of example 29, wherein the lead retention device is configured to control the length of the electrical lead within the vasculature by securing the excess portion of the electrical lead within the inner volume of the expandable member in a storage configuration.

Example 31: the device of any of examples 29 and 30, wherein the expandable member is configured to isolate the implantable medical device and the excess portion of the electrical lead from tissue of the patient.

Example 32: the device of example 31, wherein the second portion of the expandable member is configured to isolate the inner volume of the expandable member from the tissue of the patient when disposed over the first portion.

Example 33: the device of any of examples 29-32, wherein the expandable member is configured to roll over implantable medical device from a distal end of the implantable medical device to a proximal end of the implantable medical device.

Example 34: the device of any of examples 29-33, wherein the second portion of the expandable member is configured to be disposed over the first portion through twisting of the second portion relative to the first portion and rolling of the second portion over the first portion.

Example 35: the device of any of examples 29-34, wherein the expandable member comprises silicone.

Example 36: the device of any of examples 29-34, wherein the expandable member comprises a biocompatible polymer.

Example 37: the device of any of examples 29-36, wherein the expandable member further comprises an attachment member connected to a distal end of the first portion of the expandable member, wherein the attachment member is configured to secure the expandable member to at least one of the implantable medical device or the electrical lead.

Example 38: the device of example 37, wherein the attachment member is further configured to anchor the electrical lead within the vasculature.

Example 39: the device of any of examples 29-38, wherein the expandable member further comprises an anti-infection material.

Example 40: the device of any of examples 29-39, wherein the expandable member is configured to be self-sealing.

Example 41: the device of any of examples 29-40, wherein the expandable member comprises an anti-bleeding and anti-coagulation material.

Various examples have been described herein. Any combination of the described operations or functions is contemplated. These and other examples are within the scope of the following claims.

1. A system comprising:
an electrical lead;
an implantable medical device, wherein the electrical lead is configured to be electrically connected to the implantable medical device, and wherein the implantable medical device is configured to deliver an electrical therapy to tissue of a patient via the electrical lead; and
an expandable member configured to be disposed over the implantable medical device and an excess portion of the electrical lead, the expandable member comprising:
a first portion defining an inner volume configured to retain the implantable medical device and the excess portion of the electrical lead; and
a second portion connected to a proximal end of the first portion, the second portion configured to be disposed over at least a part of the first portion of the expandable member,
wherein the expandable member is configured to control a length of the electrical lead within vasculature of the patient.

2. The system of claim 1, wherein the expandable member is configured to control the length of the electrical lead by securing the excess portion of the electrical lead within the inner volume in a storage configuration.

3. The system of claim 1, wherein the expandable member is configured to isolate the implantable medical device and the excess portion of the electrical lead from the tissue of the patient.

4. The system of claim 3, wherein the second portion of the expandable member is configured to isolate the inner volume of the expandable member from the tissue of the patient when disposed over the first portion.

5. The system of claim 1, wherein the expandable member is configured to roll over the implantable medical device from a distal end of the implantable medical device to a proximal end of the implantable medical device.

6. The system of claim 1, wherein the second portion of the expandable member is configured to be disposed over the first portion through twisting of the second portion relative to the first portion and rolling of the second portion over the first portion.

7. (canceled)

8. The system of claim 1, wherein the expandable member comprises a biocompatible polymer.

9. The system of claim 1, wherein the expandable member further comprises an attachment member connected to a distal end of the first portion, wherein the attachment member is configured to secure the expandable member to at least one of the implantable medical device or the electrical lead.

10. The system of claim 9, wherein the attachment member is further configured to anchor the electrical lead within the vasculature of the patient.

11. The system of claim 1, wherein the expandable member further comprises an anti-infection material.

12. (canceled)

13. The system of claim 1, wherein the expandable member is self-sealing.

14. The system of claim 1, wherein the expandable member comprises an anti-bleeding and anti-coagulation material.

15. A method comprising:
inserting a distal portion of an electrical lead of a medical device system into vasculature of a patient, wherein the medical device system comprises:
an implantable medical device electrically connected to the electrical lead;
an expandable member comprising:
a first portion defining an inner volume; and
a second portion connected to a proximal end of the first portion;
navigating the distal portion of the electrical lead to a target location within the vasculature of the patient;
implanting the distal portion of the electrical lead in tissue at the target location;
disposing the implantable medical device and an excess portion of the electrical lead within the inner volume of the expandable member;
disposing the second portion of the expandable member over at least a part of the first portion of the expandable member; and
delivering electrical therapy to the tissue at the target location through the electrical lead.

16. The method of claim 15, wherein reducing the length of the electrical lead to the desired length comprises:
determining the desired length and the excess portion of the electrical lead;
securing the excess portion of the electrical lead into a storage configuration; and
storing the excess portion of the electrical lead in the inner volume of the expandable member in the storage configuration.

17. The method of claim 15, wherein the expandable member isolates the implantable medical device and the excess portion of the electrical lead from the tissue of the patient.

18. The method of claim 15, wherein disposing the implantable medical device and the excess portion of the electrical lead within the inner volume of the expandable member comprises rolling the expandable member over the implantable medical device from a distal end of the implantable medical device to a proximal end of the implantable medical device.

19. The method of claim 15, wherein disposing the second portion of the expandable member over the first portion of the expandable member comprises:
twisting the second portion relative to the first portion; and
roll the second portion over the first portion from the proximal end of the first portion towards a distal end of the first portion.

20. A lead retention device comprising:
an expandable member comprising:
a first portion defining an inner volume, wherein the inner volume is configured to retain an implantable medical device and an excess portion of an electrical lead; and
a second portion connected to a proximal end of the first portion, wherein the second portion is configured to be disposed over at least a part of the first portion of the expandable member,
wherein the lead retention device is configured to control a length of the electrical lead within vasculature of a patient.

21. The lead retention device of claim 20, wherein the lead retention device is configured to control the length of the electrical lead within the vasculature by securing the excess portion of the electrical lead within the inner volume of the expandable member in a storage configuration.

22. The lead retention device of claim 20, wherein the second portion of the expandable member is configured to be disposed over the first portion through twisting of the second portion relative to the first portion and rolling of the second portion over the first portion.
